(19) United States
(12) Patent Application Publication
(10) Pub. No.: US 2018/0245149 A1
(43) Pub. Date: Aug. 30, 2018

(54) FABRICATION OF A DEVICE FOR SINGLE-MOLECULE DNA SEQUENCING USING SIDEWALL LITHOGRAPHY

(71) Applicant: SEAGATE TECHNOLOGY LLC, Cupertino, CA (US)

(72) Inventors: ShuaiGang XIAO, Fremont, CA (US); David S. KUO, Palo Alto, CA (US); Kim Yang LEE, Fremont, CA (US); Xiaomin YANG, Livermore, CA (US); Koichi WAGO, Sunnyvale, CA (US); Thomas Young CHANG, Menlo Park, CA (US)

(73) Assignee: SEAGATE TECHNOLOGY LLC, Cupertino, CA (US)

(21) Appl. No.: 15/886,560

(22) Filed: Feb. 1, 2018

Related U.S. Application Data
(60) Provisional application No. 62/453,323, filed on Feb. 1, 2017.

(51) Int. Cl. C12Q 1/6874; G06F 19/22; G01N 27/447; B82Y 40/00

(52) U.S. Cl. CPC C12Q 1/6874; B82Y 40/00; G01N 27/44791; G06F 19/22

(57) ABSTRACT

A nanochannel DNA sequencing device and related methods of fabrication and of DNA sequencing are provided. In one example, a device may include a nanochannel having a width of no greater than about 2 nm and a height no greater than 1.5 times the width. The device may further include a pair of electrodes having a width of no greater than about 10 nm, the electrodes being exposed within the nanochannel to measure electronical characteristics of a DNA strand passing through the nanochannel. In one example, the nanochannel may be formed using lithography techniques, such as sidewall lithography.

### RELATED APPLICATIONS

This application claims the benefit of the filing date of U.S. Provisional Application No. 62/453,323, filed on 1 Feb. 2017, and entitled FABRICATION OF A DEVICE FOR SINGLE-MOLECULE DNA SEQUENCING USING SIDEWALL LITHOGRAPHY, the disclosure of which is incorporated in its entirety by this reference.

### SUMMARY

One aspect of the present disclosure relates to a nanochannel DNA sequencing device that includes a nanochannel having a width of no greater than about 2 nm and a height no greater than 1.5 times the width, and a pair of electrodes having a width of no greater than about 10 nm. The electrodes are exposed within the nanochannel to measure a DNA strand passing through the nanochannel.

The nanochannel width may be no greater than about 1 nm. The electrode width may be no greater than about 5 nm. The electrode width may be no greater than about 1 nm. The electrode width may be no greater than about 0.5 nm. The nanochannel and the electrodes may be oriented in a common plane. The nanochannel and the electrodes may be oriented such that they are substantially orthogonal to each other. The device may be formed using a lithography process, such as a sidewall lithography process.

Another aspect of the present disclosure relates to a method of forming a nanochannel device for DNA sequencing. The method includes providing a substrate and depositing a first sacrificial layer over the substrate, the first sacrificial layer extending across a portion of a width of the substrate and having an exposed sidewall. The method further includes depositing a second sacrificial layer on the substrate and the first sacrificial layer, the second sacrificial layer covering the exposed sidewall, and then etching the first and second sacrificial layers to form a channel deposit. An electrode is formed over the substrate using, for example, a subsequent lithography and/or metal lift-off process. A spin on glass (SOG) coating is then deposited over the substrate. The SOG coating is etched back and the channel deposit is removed using, for example, either a dry or a wet etch process.

The method may include providing an insulator layer on the substrate, the first sacrificial layer positioned on the insulator layer. The first sacrificial layer may include a photoresist layer, and the second sacrificial layer may include Chromium (Cr). The second sacrificial layer may be formed by one of sputter deposition, chemical vapor deposition, and atomic layer deposition. The channel deposit may have a width in the range of about 0.5 nm to about 1 nm. Removing the channel deposit may include removing using dry reactive ion etching (RIE) or wet chemical etching. The method may include depositing an insulation coating after removing the channel deposit. Depositing the insulation coating may include depositing by isotropic deposition.

Another aspect of the present disclosure relates to a method of sequencing DNA. The method includes providing a device having a nanochannel exhibiting a width of no greater than about 2 nm and a height no greater than 1.5 times the width and a pair of electrodes exhibiting a width of no greater than about 10 nm, the electrodes being exposed within the nanochannel. A DNA strand is passed through the nanochannel and electrical characteristics of individual nucleotides of the DNA strand are measured with the electrodes as the DNA strand passes through the nanochannel. The method may also include determining a sequence of the nucleotides based on the electronic signals.

The method may further include providing the device with a pair of ion electrodes to drive the DNA strand through the nanochannel with electrophoresis. The act of measuring electrical characteristics may include measuring transverse electron current. The method may further include configuring at least a portion of the nanochannel to exhibit a width of no greater than about 1 nm.

The foregoing has outlined rather broadly the features and technical advantages of examples according to this disclosure so that the following detailed description may be better understood. Additional features and advantages will be described below. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed herein, including their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the present disclosure may be realized by reference to the following drawings. In the appended figures, similar components or features may have the same reference label. Further, various components of the same type may be distinguished by following a first reference label with a dash and a second label that may distinguish among the similar components. However, features discussed for various components—including those having a dash and a second reference label—apply to other similar components. If only the first reference label is used in the specification, the description is applicable to any one of the similar components having the same first reference label irrespective of the second reference label.

FIG. 1 is a perspective view of a single-molecule DNA sequencing device according to the present disclosure.

FIG. 2 is a perspective view of another single-molecule DNA sequencing device according to the present disclosure.

FIGS. 3A-3H show fabrication steps for a single-molecule DNA sequencing device using sidewall lithography.

FIG. 4 shows a diagram of a system in accordance with various aspects of this disclosure.

FIG. 5 shows steps of a fabrication process of a top-bottom electrode pair in accordance with the present disclosure.

FIG. 6 shows steps of a fabrication process of another top-bottom electrode pair in accordance with the present disclosure.

### DETAILED DESCRIPTION

The present disclosure generally relates to DNA sequencing, and more particularly relates to DNA sequencing devices having nanochannels and nanoelectrodes, and related methods of fabricating such devices. The present disclosure may also relate to DNA sequencing using such devices.

The present disclosure also relates to DNA sequencing using nanofluidics. Various methods are disclosed herein for fabricating a DNA sequencing device that includes a nanochannel and a pair of in-plane nanoelectrodes. In one example, the nanochannel and nanoelectrodes are formed using sidewall lithography methods. The disclosed devices and methods provide solutions to the DNA sequencing challenges of high throughput, long read length, and low cost.

Despite considerable efforts, DNA sequencing currently still suffers from high costs and low speeds. To address all these issues, various methods have been proposed over the past decade that would enable individual DNA strands to be read directly. Among these, nanopore and nanochannel based approaches have emerged as the most promising. However, many challenges exist related to fabricating a channel and/or pore opening that is sufficiently small to limit passage to a single DNA strand, and there is currently no such report of a relatively mature method or technology that addresses this unmet need.

Direct DNA sequencing has drawn attention due to its advantages on long read length, high throughput and low cost. Direct DNA sequencing methods using transverse tunneling current measurement have been studied extensively in literature. However, a manufacturably viable direct DNA sequencing device with required dimensions for the gap between the nanoelectrodes, nor methods for creating such a device, have not been discovered. Conventional MEMS and nanofabrication methods are inadequate for creating the required structure. No manufacturable (for mass production) method has been reported for the fabrication of single-molecular direct sequencing device based on transverse current measurement.

Currently, in a conventional DNA sequencing process, by using the so-called second generation sequencing technologies, double-stranded DNA is fragmented by enzymes and ultrasonication. Then a polymerase chain reaction (PCR) technique is utilized to amplify the fragmented DNA since single DNA modified with fluorescent dye is unable to be detected. This process is time consuming and expensive. These methods typically can only be used for a short read of a DNA molecular/segment with a few hundred bases due to the problem of gradual intermolecular dephasing.

To meet one or more of the challenges, for example, of (1) high throughput, (2) long read length, and (3) low cost, nanopore- or nanochannel-based sequencing method and related techniques are highly desired. A nanopore/nanochannel device can, at least, employ longitudinal (e.g., relative to the backbone of DNA molecule) ion current, or transverse tunneling current for single-molecule detection.

DNA is electrophoretically driven through the channel owing to two ion electrodes which can be operated at alternating +/− voltages for multiple tests. A very narrow nanochannel will ensure the translocation of a ssDNA in the center of the channel, and a pair of transverse electrodes are employed to measure tunneling current which will be changed during the passing of the DNA molecule.

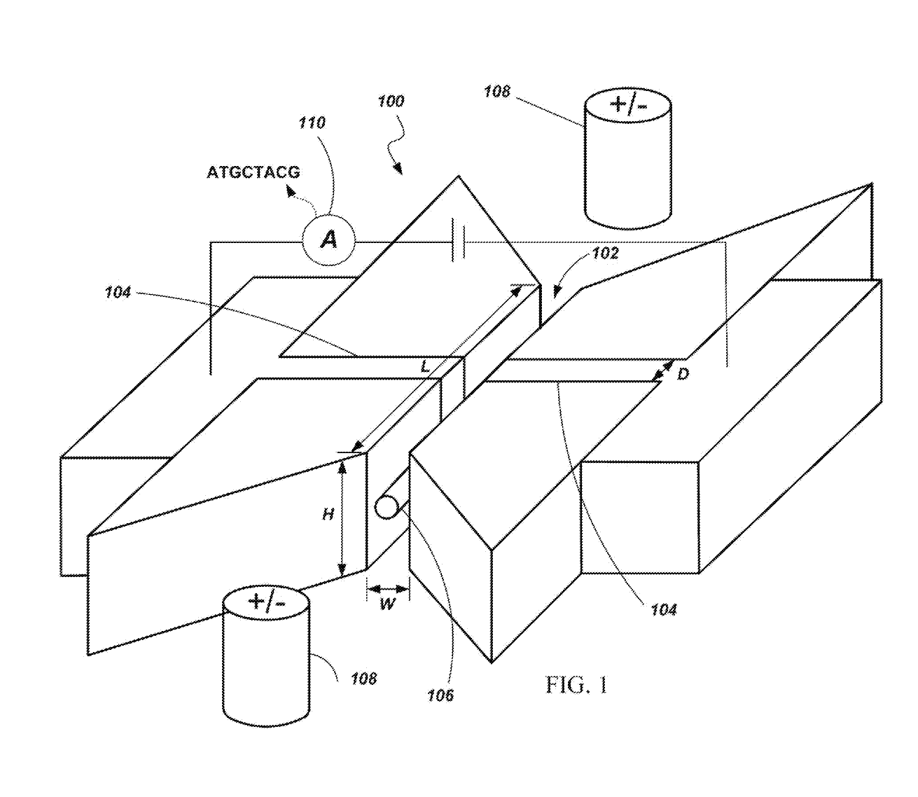

The present disclosure provides a nanochannel sequencing device by measuring transverse electronic tunneling current. Referring to FIG. 1, a 3D schematic drawing illustrates a DNA sequencing device 100 having a nanochannel 102 and a pair of nanoelectrodes 104. In accordance with one embodiment of the disclosure, at least three dimensions should to be controlled in providing such a device 100, including: (1) channel width W to a dimension in a range of about 0.1 nm to about 2 nm and, in one embodiment, no greater than about 1 nm, to control the orientation/position of the single DNA strand 106 in the channel; (2) transverse electrode width D to a dimension in a range of less than about 5 nm to about 10 nm for single-molecule detection only, or less than about 0.5 nm to about 1 nm for single-base spatial resolution (single base length equal to about 0.3 nm to about 0.4 nm); and (3) channel height H to a dimension of greater than or equal to the channel width W, but not much larger than W, to minimize background noise from fluid during transverse current measurement (e.g., a ratio of H to W being approximately 1:1 to approximately 1:1.5).

The disclosed devices may include, for example, two structural features that contribute to the advantages provided by the device and related fabrication methods: (1) a nanochannel 102 with a relatively small width W (e.g., in the range of about 1 nm or less), and a relatively small height H that is comparable to the width W or not much larger (such as noted above), and (2) a pair of electrodes 104 with a relatively small width D (e.g., less than about 5 nm to about 10 nm, or even less than about 0.5 nm to about 1 nm). A patterning method called sidewall lithography may be used to obtain precisely-controlled critical dimensions (CDs) of as small as about 1 nm or less. This method may be particularly useful for defining the width (W) of nanochannel 102 and the width (D) of nanoelectrodes 104.

During operation, ion electrodes 108 may be used to motivate the DNA strand 106 (disposed in a fluid solution) through the nanochannel 102 and past the electrodes 104, such that the electrode records desired electrical characteristics of the DNA strand 106 (e.g., by measuring transverse electron current while the DNA passes by the electrodes) to provide a sequencing of the DNA strand 106 as indicated at 110.

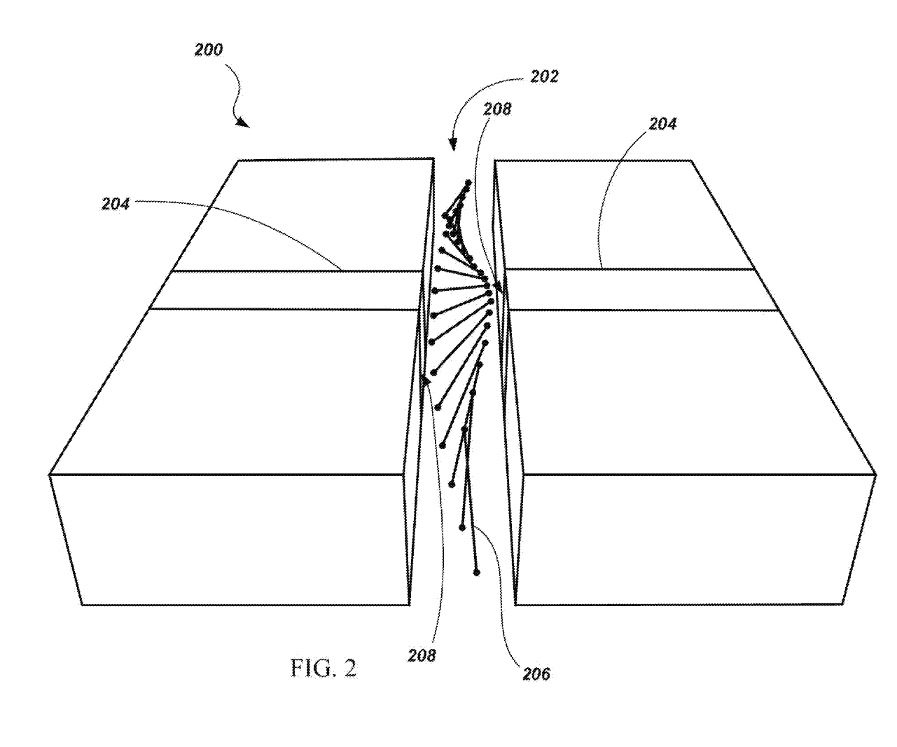

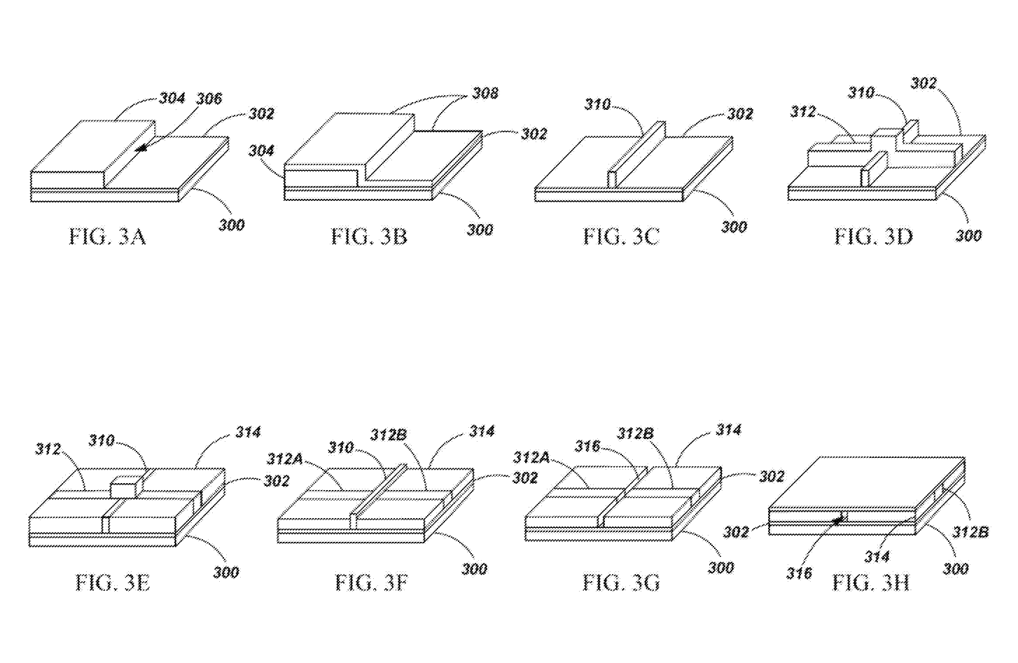

FIG. 2 shows a 3D schematic drawing of a structure 200 having an in-plane nanochannel 202 together with two in-plane nanoelectrodes 204. The structure 204 may be used, for example, in the DNA sequencing device 100 describe above and may exhibit dimensions such as described above for use in sequencing a DNA strand 206. Forming the nanoelectrodes 204 in-plane with the nanochannel 202 such that a face 208 or other portion of the nanoelectrodes 204 are exposed within the nanochannel 202 provides many fabrication challenges. An example fabrication method disclosed with reference to FIGS. 3A-3H addresses many of these challenges and meets the design requirements and functionality related to DNA sequencing as described herein. Other methods and/or method steps and/or processes may be used to provide similar results.

Referring to FIG. 3A, an initial step includes providing a substrate 300 which, in one embodiment, may include an insulative substrate. In other embodiments, an optional insulating layer 302 may be disposed over a surface of the substrate. For example, the substrate 300 may be formed of silicon and an insulative layer 302 may be formed of a material such as silicon dioxide (SiO₂). A first sacrificial layer 304 may be disposed over the substrate 300 and/or the sacrificial layer 302 such that it extends across a portion of a width of the substrate and provides an exposed sidewall 306. The first sacrificial layer 304 may comprise carbon, or a photoresist or another similar material.

As shown in FIG. 3B, a second sacrificial layer 308 is conformally deposited on the first sacrificial layer 304 and a portion of the substrate 300 (and/or the insulating layer 302). The second sacrificial layer covers the exposed sidewall 306. In one embodiment, the second sacrificial layer 308 may comprise chromium (Cr). Other potential materials from which the second sacrificial layer 308 may be formed include, for example, tantalum (Ta), titanium dioxide (TiO₂) or other metallic or non-metallic materials. The second sacrificial layer 308 may be deposited by, for example, sputtering, chemical vapor deposition, atomic layer deposition, or other similar techniques.

The first and second sacrificial layers 304 and 308 are etched to form a channel deposit or core member 310 as shown in FIG. 3C. In one embodiment, the channel deposit or core member 310 may be formed of a material such as Cr or similar material.

A subsequent step includes forming an electrode member 312 on the substrate as shown in FIG. 3D. In one embodiment, the electrode member 312 may be formed of a metal material. It is noted that the metal, or metal materials, in this description may generally refer to a conductor or an electronically conductive material, and may include any desired conductive material. When formed using sidewall lithography, the nanoelectrode may be formed with a width (see, e.g., width D in FIG. 1) in the range of less than about 0.5 nm to about 10 nm.

A spin on glass (SOG) coating 314 is then applied to the device as shown in FIG. 3E, followed by etching back the SOG coating 314 as shown in FIG. 3F, and removing the channel deposit or core member 310, leaving a nanochannel 316 and an electrode pair 312A and 312B formed in the remaining SOG coating 314 as shown in FIG. 3G. The sacrificial material used to form the channel deposit 310 may be removed, for example, by dry reactive ion etching (RIE) or wet chemical etch.

As seen in FIG. 3H, an optional step may include covering the nanochannel 316 by deposition of an insulating layer through, for example, an isotropic deposition process to close over the top of the nanochannel 316 without filling it with the insulative material.

While sidewall lithography has been used as an example to form the electrodes in the method process shown and described with respect to FIGS. 3A-3H, it is noted that other processes may also be used. For example, conventional lithography (e.g., electron-beam, etc.) along with either additive (e.g., liftoff, etc.) or subtractive (e.g., etching, etc.) pattern transfer processes may be used for widths D in the range of less than about 5 nm to about 10 nm.

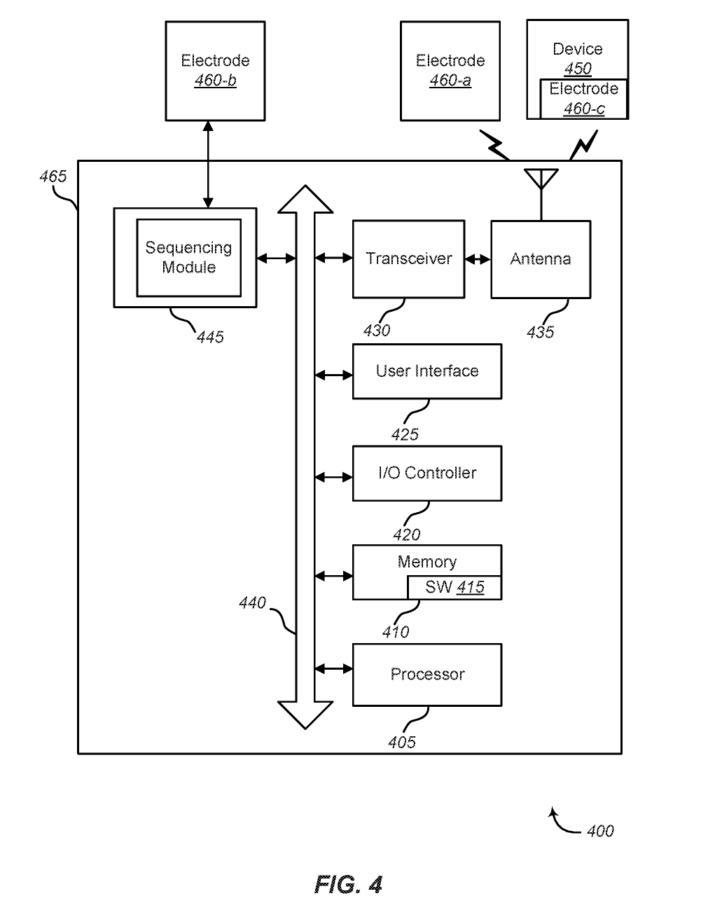

FIG. 4 shows a system 400 for use with the DNA sequencing device 100 shown in FIG. 1, or the systems and/or devices shown in FIGS. 2 and 3. System 400 may include a control panel 465. Control panel 465 may be equivalent at least in part to a controller, control unit, processor or the like for use with the devices described above with reference to FIGS. 1-3. Control panel 465 may include sequencing module 445. The sequencing module 445 may provide communications with one or more electrodes 460 (also referred to as sensors or devices) directly or via other communication components, such as a transceiver 430 and/or antenna 435. The electrodes 460 may represent one or more of the electrodes 104 or pairs of such electrodes described above. The sequencing module 445 may perform or control various operations associated with, for example, the electrodes 104, energy source 108, controller, or other components of the DNA sequencing devices and related systems as described above with reference to FIGS. 1-3.

Control panel 465 may also include a processor module 405, and memory 410 (including software/firmware code (SW) 415), an input/output controller module 420, a user interface module 425, a transceiver module 430, and one or more antennas 435 each of which may communicate, directly or indirectly, with one another (e.g., via one or more buses 440). The transceiver module 430 may communicate bi-directionally, via the one or more antennas 435, wired links, and/or wireless links, with one or more networks or remote devices. For example, the transceiver module 430 may communicate bi-directionally with one or more of device 450 and/or electrodes 460-a, 460-c. The device 450 may be components of the DNA sequencing device 100 and related systems and devices described with reference to FIGS. 1-3, or other devices in communication with such systems and devices. The transceiver 430 may include a modem to modulate the packets and provide the modulated packets to the one or more antennas 435 for transmission, and to demodulate packets received from the one or more antennas 435. In some embodiments (not shown) the transceiver may be communicate bi-directionally with one or more of device 450, remote control device 455, and/or electrodes 460-a, 460-c through a hardwired connection without necessarily using antenna 435. While a control panel or a control device (e.g., 405) may include a single antenna 435, the control panel or the control device may also have multiple antennas 435 capable of concurrently transmitting or receiving multiple wired and/or wireless transmissions. In some embodiments, one element of control panel 465 (e.g., one or more antennas 435, transceiver module 430, etc.) may provide a connection using wireless techniques, including digital cellular telephone connection, Cellular Digital Packet Data (CDPD) connection, digital satellite data connection, and/or another connection.

The signals associated with system 400 may include wireless communication signals such as radio frequency, electromagnetics, local area network (LAN), wide area network (WAN), virtual private network (VPN), wireless network (using 802.11, for example), 345 MHz, Z-WAVE®, cellular network (using 3G and/or LTE, for example), and/or other signals. The one or more antennas 435 and/or transceiver module 430 may include or be related to, but are not limited to, WWAN (GSM, CDMA, and WCDMA), WLAN (including BLUETOOTH® and Wi-Fi), WMAN (WiMAX), antennas for mobile communications, antennas for Wireless Personal Area Network (WPAN) applications (including RFID and UWB). In some embodiments, each antenna 435 may receive signals or information specific and/or exclusive to itself. In other embodiments, each antenna 435 may receive signals or information not specific or exclusive to itself.

In some embodiments, one or more electrodes 460 (e.g., voltage, inductance, resistance, current, force, temperature, etc.) may connect to some element of system 400 via a network using one or more wired and/or wireless connections. In some embodiments, the user interface module 425 may include an audio device, such as an external speaker system, an external display device such as a display screen, and/or an input device (e.g., remote control device interfaced with the user interface module 425 directly and/or through I/O controller module 420).

One or more buses 440 may allow data communication between one or more elements of control panel 465 (e.g., processor module 405, memory 410, I/O controller module 420, user interface module 425, etc.).

The memory 410 may include random access memory (RAM), read only memory (ROM), flash RAM, and/or other types. The memory 410 may store computer-readable, computer-executable software/firmware code 415 including instructions that, when executed, cause the processor module 405 to perform various functions described in this disclosure (e.g., initiating an adjustment of a lighting system, etc.). Alternatively, the software/firmware code 415 may not be directly executable by the processor module 405 but may cause a computer (e.g., when compiled and executed) to perform functions described herein. Alternatively, the computer-readable, computer-executable software/firmware code 415 may not be directly executable by the processor module 405 but may be configured to cause a computer (e.g., when compiled and executed) to perform functions described herein. The processor module 405 may include an intelligent hardware device, e.g., a central processing unit (CPU), a microcontroller, an application-specific integrated circuit (ASIC), etc.

In some embodiments, the memory 410 can contain, among other things, the Basic Input-Output system (BIOS) which may control basic hardware and/or software operation such as the interaction with peripheral components or devices. For example, the resistance module 445, and other modules and operational components of the control panel 465 used to implement the present systems and methods may be stored within the system memory 410. Applications resident with system 400 are generally stored on and accessed via a non-transitory computer readable medium, such as a hard disk drive or other storage medium. Additionally, applications can be in the form of electronic signals modulated in accordance with the application and data communication technology when accessed via a network interface (e.g., transceiver module 430, one or more antennas 435, etc.).

Many other devices and/or subsystems may be connected to one or may be included as one or more elements of system 400. In some embodiments, all of the elements shown in FIG. 4 need not be present to practice the present systems and methods. The devices and subsystems can be interconnected in different ways from that shown in FIG. 4. In some embodiments, an aspect of some operation of a system, such as that shown in FIG. 4, may be readily known in the art and are not discussed in detail in this application. Code to implement the present disclosure can be stored in a non-transitory computer-readable medium such as one or more of system memory 410 or other memory. The operating system provided on I/O controller module 420 may be iOS®, ANDROID®, MS-DOS®, MS-WINDOWS®, OS/2®, UNIX®, LINUX®, or another known operating system.

The transceiver module 430 may include a modem configured to modulate the packets and provide the modulated packets to the antennas 435 for transmission and/or to demodulate packets received from the antennas 435. While the control panel or control device (e.g., 405) may include a single antenna 435, the control panel or control device (e.g., 405) may have multiple antennas 435 capable of concurrently transmitting and/or receiving multiple wireless transmissions.

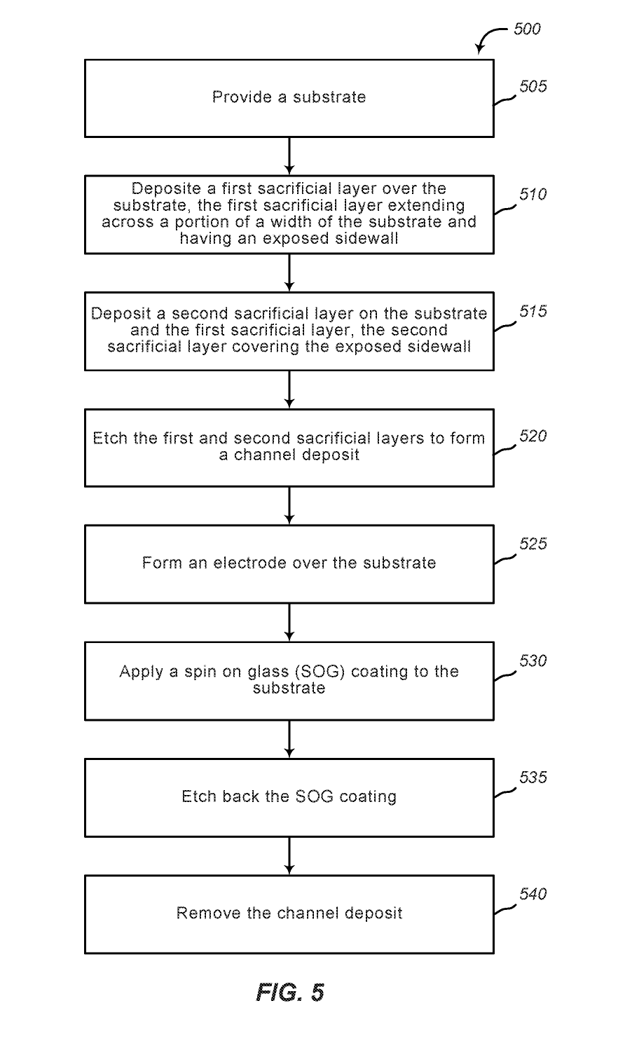

FIG. 5 is a flow chart illustrating an example of a method 500 for forming a nanochannel device for DNA sequencing, in accordance with various aspects of the present disclosure. One or more aspects of the method 500 may be implemented in conjunction with the devices and/or components 100, 200, 300 of FIGS. 1-3. In some examples, a computing device may execute one or more sets of code to control functional elements of the DNA sequencing devices disclosed herein to perform one or more of the functions described below. Additionally, or alternatively, computing devices and/or storage devices may perform one or more of the functions described below using special-purpose hardware.

The method 500 may include, at block 505, providing a substrate. Block 510 includes depositing a first sacrificial layer over the substrate, the first sacrificial layer extending across a portion of a width of the substrate and having an exposed sidewall. Block 515 includes depositing a second sacrificial layer on the substrate and the first sacrificial layer, the second sacrificial layer covering the exposed sidewall. Block 520 includes etching the first and second sacrificial layers to form a channel deposit. Block 525 includes forming an electrode over the substrate. Block 530 includes applying a spin on glass (SOG) coating to the substrate. Block 535 includes etching back the SOG coating. Block 540 includes removing the channel deposit.

The method 500 may also include providing an insulator layer on the substrate, the first sacrificial layer positioned on the insulator layer. The first sacrificial layer may include carbon or a photoresist material. The second sacrificial layer may include Chromium (Cr). The second sacrificial layer may be formed by one of sputter deposition, chemical vapor deposition, and atomic layer deposition. The channel deposit may have a width in the range of about 0.5 nm to about 1 nm. Removing the channel deposit may include using dry reactive ion etching (RIE) or wet chemical etching. Method 500 may further include depositing an insulation coating on the electrode and any remaining SOG material after removing the channel deposit. Depositing the insulation coating may include depositing by isotropic deposition.

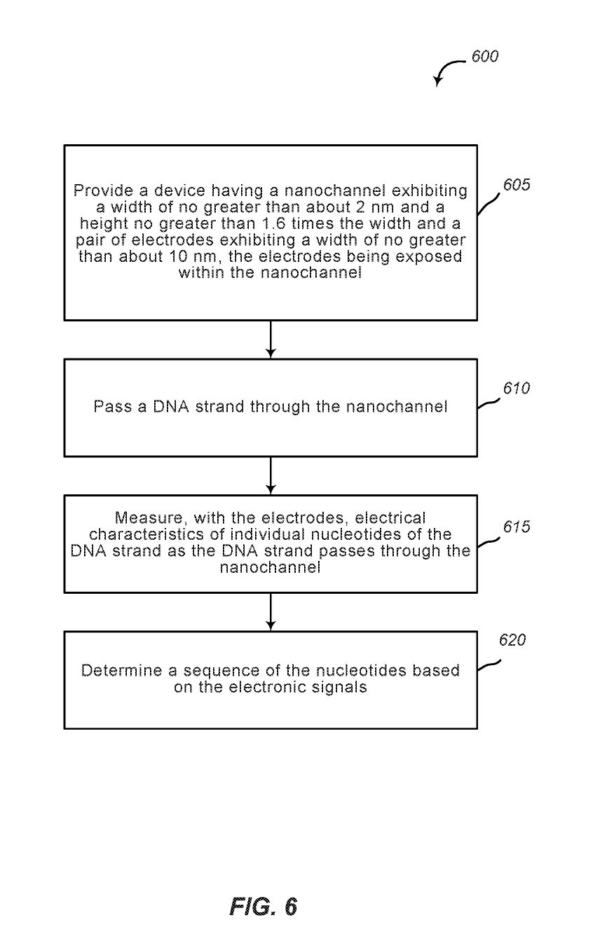

FIG. 6 is a flow chart illustrating an example of a method 600 for DNA sequencing device, in accordance with the various aspects of the present disclosure. One or more aspects of the method 600 may be implemented in conjunction with the devices and/or components 100, 200, 300 described with reference to FIGS. 1-3. In some examples, a computing device may execute one or more sets of code to control functional elements of the DNA sequencing device as disclosed herein to perform one or more of the functions described below. Additionally, or alternatively, computing devices and/or storage devices may perform one or more of the functions described below using special purpose hardware.

The method 600 may include, at block 605, providing a device having a nanochannel exhibiting a width of no greater than about 2 nm and a height no greater than 1.5 times the width and a pair of electrodes exhibiting a width of no greater than about 10 nm, the electrodes being exposed within the nanochannel. The method 600 also includes passing a DNA strand through the nanochannel, measuring, with the electrodes, electrical characteristics of individual nucleotides of the DNA strand as the DNA strand passes through the nanochannel, and determining a sequence of the nucleotides based on the electronic signals.

The method 600 may further include providing the device with a pair of ion electrodes to motivate the DNA strand through the nanochannel, and measuring electrical characteristics includes measuring transverse electron current. The method 600 may further configuring the nanochannel to exhibit a width of no greater than about 1 nm.

The example methods 500, 600 may, in other embodiments, include fewer or additional steps that those illustrated in FIGS. 5 and 6. Further, many other methods and method steps may be possible based on the disclosures provided herein.

In some embodiments, the DNA sequencing device and systems described herein may be used to collect electronic signals associated with the nucleotides of a DNA strand passing through the gap between electrode pairs, and the collected electronic signals are processed at a different location. The processing may include electronically comparing the collected electronic signals to ranges of electronic signals associated with specific nucleotide types which have been previously determined and stored. In other embodiments, the DNA sequencing device includes capability of processing the collected electronic signals, conducting such comparison evaluations, and even formulating an order or sequence for the nucleotides of the DNA strand being evaluated.

### INCORPORATION BY REFERENCE

The entire content of each of the previously filed provisional patent applications listed below are incorporated by reference in their entireties into this document, as are the related non-provisional patent applications of the same title filed concurrently with the present application. If the same term is used in both this document and one or more of the incorporated documents, then it should be interpreted to have the broadest meaning imparted by any one or combination of these sources unless the term has been explicitly defined to have a different meaning in this document. If there is an inconsistency between any of the following documents and this document, then this document shall govern. The incorporated subject matter should not be used to limit or narrow the scope of the explicitly recited or depicted subject matter.
U.S. Prov. App. No. 62/453,270, titled “SINGLE-MOLECULE DNA SEQUENCING METHOD USING CONFINED NANO-FLUIDIC CHANNEL AND SUB-NANOMETER ELECTRODE GAP,” filed on 1 Feb. 2017, and U.S. patent application Ser. No. ______, titled “SINGLE-MOLECULE DNA SEQUENCING METHOD USING CONFINED NANO-FLUIDIC CHANNEL AND SUB-NANOMETER ELECTRODE GAP,” filed on 1 Feb. 2018.
U.S. Prov. App. No. 62/453,398, titled “NANOFLUIDIC CHANNEL OPENING SIZE CONTROL USING ACTUATION,” filed on 1 Feb. 2017, and U.S. patent application Ser. No. ______, titled “NANOFLUIDIC CHANNEL OPENING SIZE CONTROL USING ACTUATION,” filed on 1 Feb. 2018.
U.S. Prov. App. No. 62/453,298, titled “FABRICATION OF NANOCHANNEL

WITH INTEGRATED ELECTRODES FOR DNA SEQUENCING USING TUNNELING CURRENT,” filed on 1 Feb. 2017, and U.S. patent application Ser. No. ______, titled “FABRICATION OF NANOCHANNEL WITH INTEGRATED ELECTRODES FOR DNA SEQUENCING USING TUNNELING CURRENT,” filed on 1 Feb. 2018.
U.S. Prov. App. No. 62/453,307, titled “METHOD TO FABRICATE A NANOCHANNEL FOR DNA SEQUENCING BASED ON NARROW TRENCH PATTERNING PROCESS,” filed on 1 Feb. 2017, and U.S. patent application Ser. No. ______, titled “METHOD TO FABRICATE A NANOCHANNEL FOR DNA SEQUENCING BASED ON NARROW TRENCH PATTERNING PROCESS,” filed on 1 Feb. 2018.
U.S. Prov. App. No. 62/453,339, titled “FABRICATION OF A NANOCHANNEL FOR DNA SEQUENCING USING ELECTRICAL PLATING TO ACHIEVE TUNNELING ELECTRODE GAP,” filed on 1 Feb. 2017, and U.S. patent application Ser. No. ______, titled “FABRICATION OF A NANOCHANNEL FOR DNA SEQUENCING USING ELECTRICAL PLATING TO ACHIEVE TUNNELING ELECTRODE GAP,” filed on 1 Feb. 2018.
U.S. Prov. App. No. 62/453,346, titled “NANOSTRUCTURES TO CONTROL DNA STRAND ORIENTATION AND POSITION LOCATION FOR TRANSVERSE DNA SEQUENCING,” filed on 1 Feb. 2017, and U.S. patent application Ser. No. ______, titled “NANOSTRUCTURES TO CONTROL DNA STRAND ORIENTATION AND POSITION LOCATION FOR TRANSVERSE DNA SEQUENCING,” filed on 1 Feb. 2018.
U.S. Prov. App. No. 62/453,365, titled “FABRICATION OF WEDGE SHAPED ELECTRODE FOR ENHANCED DNA SEQUENCING USING TUNNELING CURRENT,” filed on 1 Feb. 2017, and U.S. patent application Ser. No. ______, titled “FABRICATION OF WEDGE SHAPED ELECTRODE FOR ENHANCED DNA SEQUENCING USING TUNNELING CURRENT,” filed on 1 Feb. 2018.
U.S. Prov. App. No. 62/453,329, titled “DIRECT SEQUENCING DEVICE WITH

A TOP-BOTTOM ELECTRODE PAIR,” filed on 1 Feb. 2017, and U.S. patent application Ser. No. ______, titled “DIRECT SEQUENCING DEVICE WITH A TOP-BOTTOM ELECTRODE PAIR,” filed on 1 Feb. 2018.
U.S. Prov. App. No. 62/453,376, titled “MICRO AND NANOFLUIDIC CHANNEL CONTROLLED ACTUATION TO OPEN CHANNEL GAP,” filed on 1 Feb. 2017.
U.S. Prov. App. No. 62/469,393, titled “METHOD TO AMPLIFY TRANSVERSE TUNNELING CURRENT DISCRIMINATION OF DNA NUCLEOTIDES VIA NUCLEOTIDE SITE SPECIFIC ATTACHMENT OF DYE-PEPTIDE,” filed on 9 Mar. 2017, and U.S. patent application Ser. No. ______, titled “METHOD TO AMPLIFY TRANSVERSE TUNNELING CURRENT DISCRIMINATION OF DNA NUCLEOTIDES VIA NUCLEOTIDE SITE SPECIFIC ATTACHMENT OF DYE-PEPTIDE,” filed on 9 Mar. 2018.
U.S. Prov. App. No. 62/469,409, titled “VERTICAL NANOPORE COUPLED WITH A PAIR OF TRANSVERSE ELECTRODES HAVING A UNIFORM ULTRASMALL NANOGAP FOR DNA SEQUENCING,” filed on 9 Mar. 2017, and U.S. patent application Ser. No. ______, titled “VERTICAL NANOPORE COUPLED WITH A PAIR OF TRANSVERSE ELECTRODES HAVING A UNIFORM ULTRASMALL NANOGAP FOR DNA SEQUENCING,” filed on 9 Mar. 2018.

The detailed description set forth above in connection with the appended drawings describes examples and does not represent the only instances that may be implemented or that are within the scope of the claims. The terms “example” and “exemplary,” when used in this description, mean “serving as an example, instance, or illustration,” and not “preferred” or “advantageous over other examples.” The detailed description includes specific details for the purpose of providing an understanding of the described techniques. These techniques, however, may be practiced without these specific details. In some instances, known structures and apparatuses are shown in block diagram form in order to avoid obscuring the concepts of the described examples.

As used herein, including in the claims, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination. Also, as used herein, including in the claims, “or” as used in a list of items (for example, a list of items prefaced by a phrase such as “at least one of” or “one or more of”) indicates a disjunctive list such that, for example, a list of “at least one of A, B, or C” means A or B or C or AB or AC or BC or ABC (i.e., A and B and C).

In addition, any disclosure of components contained within other components or separate from other components should be considered exemplary because multiple other architectures may potentially be implemented to achieve the same functionality, including incorporating all, most, and/or some elements as part of one or more unitary structures and/or separate structures.

The previous description of the disclosure is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed.

The process parameters, actions, and steps described and/or illustrated in this disclosure are given by way of example only and can be varied as desired. For example, while the steps illustrated and/or described may be shown or discussed in a particular order, these steps do not necessarily need to be performed in the order illustrated or discussed. The various exemplary methods described and/or illustrated here may also omit one or more of the steps described or illustrated here or include additional steps in addition to those disclosed.

This description, for purposes of explanation, has been described with reference to specific embodiments. The illustrative discussions above, however, are not intended to be exhaustive or limit the present systems and methods to the precise forms discussed. Many modifications and variations are possible in view of the above teachings. The embodiments were chosen and described in order to explain the principles of the present systems and methods and their practical applications, to enable others skilled in the art to utilize the present systems, apparatus, and methods and various embodiments with various modifications as may be suited to the particular use contemplated.

What is claimed is:

1. A nanochannel DNA sequencing device, comprising:
a nanochannel having a width of no greater than about 2 nm and a height no greater than 1.5 times the width;
a pair of electrodes having a width of no greater than about 10 nm, the electrodes being exposed within the nanochannel to measure a DNA strand passing through the nanochannel.

2. The device of claim 1, wherein the nanochannel width is no greater than about 1 nm.

3. The device of claim 1, wherein the electrode width is no greater than about 5 nm.

4. The device of claim 1, wherein the electrode width is no greater than about 1 nm.

5. The device of claim 1, wherein the electrode width is no greater than about 0.5 nm.

6. The device of claim 1, wherein the nanochannel and the electrodes are oriented in a common plane.

7. The device of claim 1, the nanochannel and the electrodes are oriented substantially orthogonally to one another.

8. A method of forming a nanochannel device for DNA sequencing, the method comprising:
providing a substrate;
depositing a first sacrificial layer over the substrate, the first sacrificial layer extending across a portion of a width of the substrate and having an exposed sidewall;
depositing a second sacrificial layer on the substrate and the first sacrificial layer, the second sacrificial layer covering the exposed sidewall;
etching the first and second sacrificial layers to form a channel deposit;
forming an electrode over the substrate;
applying a spin on glass (SOG) coating to the substrate;
etching back the SOG coating;
removing the channel deposit.

9. The method of claim 8, further comprising providing an insulator layer on the substrate, the first sacrificial layer positioned on the insulator layer.

10. The method of claim 8, wherein the first sacrificial layer comprises carbon or a photoresist material.

11. The method of claim 10, wherein the second sacrificial layer comprises Chromium (Cr).

12. The method of claim 8, wherein the second sacrificial layer is formed by one of sputter deposition, chemical vapor deposition, and atomic layer deposition.

13. The method of claim 8, wherein the channel deposit has a width in the range of about 0.5 nm to about 1 nm.

14. The method of claim 8, wherein removing the channel deposit includes using dry reactive ion etching (RIE) or wet chemical etching.

15. The method of claim 8, further comprising depositing an insulation coating on the electrode and any remaining SOG material after removing the channel deposit.

16. The method of claim 14, wherein depositing the insulation coating includes depositing by isotropic deposition.

17. A method of sequencing DNA, the method comprising:
providing a device having a nanochannel exhibiting a width of no greater than about 2 nm and a height no greater than 1.5 times the width and a pair of electrodes exhibiting a width of no greater than about 10 nm, the electrodes being exposed within the nanochannel;
passing a DNA strand through the nanochannel;
measuring, with the electrodes, electrical characteristics of individual nucleotides of the DNA strand as the DNA strand passes through the nanochannel;
determining a sequence of the nucleotides based on the electronic signals.

18. The method of claim 17, further comprising providing the device with a pair of ion electrodes to motivate the DNA strand through the nanochannel.

19. The method of claim 17, wherein measuring electrical characteristics includes measuring transverse electron current.

20. The method of claim 17, further comprising configuring the nanochannel to exhibit a width of no greater than about 1 nm.
